(19) United States
(12) Patent Application Publication
(10) Pub. No.: US 2014/0276318 A1
(43) Pub. Date: Sep. 18, 2014

(54) ORTHOTIC DEVICE, SYSTEM AND METHODS FOR ADDRESSING FOOT DROP

(71) Applicant: FOOTMIND, INC., Alpine, UT (US)

(72) Inventor: Jonathan Robert Faux, Provo, UT (US)

(21) Appl. No.: 13/795,060

(22) Filed: Mar. 12, 2013

(51) Int. Cl. A61F 5/01

(52) U.S. Cl. CPC A61F 5/0127
USPC 602/28

(57) ABSTRACT

A drop foot brace includes a securing member configured to be secured to a leg of a user, a foot-receiving member and a tensioning component. The tensioning component may include an elongated element a rotatable tightening member for adjusting a tension in the elongated element and two or more support engagers associated with the elongated element. Each support engager may include a long, curved hook guide that receives the elongated element and that is configured to minimize deformation and breakage of the elongate element. In addition, each support engager may include a hook configured to engage a corresponding feature of a foot-receiving member. The foot-receiving member may comprise footwear, such as a shoe or boot, or it may be configured to enable us of the support member (and, thus, of the drop foot brace) without footwear that covers the top, or dorsal portion, of the foot. Thus, the drop foot brace may be used when an individual decides to go barefoot or when the individual wears flip-flops or other sandals. Methods for addressing drop foot are also disclosed.

### TECHNICAL FIELD

This disclosure relates generally to orthotic devices and, more specifically, to orthotic devices that are configured for use in treating conditions that relate to feet, including foot drop. This disclosure also relates to systems and methods for treating foot drop.

### BACKGROUND OF RELATED ART

Foot drop, which is also known as “drop foot,” is a condition in which the front part of an individual's foot, or “forefoot,” and his or her toes drop. Various causes of foot drop include weakness of the forefoot, injury to the peroneal nerve, paralysis of muscles in the anterior, or front, portion of the lower leg (i.e., the shin), or a variety of other conditions, such as a stroke, multiple sclerosis (MS), Charcot-Marie-Tooth disease, or others. The condition may be temporary or permanent, and can affect one foot or both of an individual's feet.

Because foot drop affects an individual's ability to lift his or her forefoot, it can cause difficulty walking. A person with foot drop may drag his or her toes along the ground when walking or lift his or her knee higher than normal while walking in order to prevent the foot from dragging, resulting in what is commonly referred to as “steppage gait.” These complications may make decrease the mobility of affected individuals.

While various orthotic devices are available for treating foot drop, they are often difficult and clumsy to set up and adjust. Many require specialized or modified shoes. The complexity, weight, and lack of flexibility associated with many orthotic devices make them less convenient for affected individuals.

### SUMMARY

This disclosure, in one aspect, relates to approaches for providing an orthotic device for treating foot drop or other conditions where the forefoot of the person requires additional support.

In one aspect, an orthotic device for addressing foot drop, or a “drop foot brace,” includes a securing member that is configured to secure the drop foot brace to the leg of a user. The securing member includes an interior surface and an exterior surface. The interior surface is configured to face inwardly toward the leg of the user, while the exterior surface is configured to face outwardly, away from the user's leg. The securing member may comprise an elongated element, such as a band, that is configured to be wrapped at least partially around the user's leg. In some embodiments, the securing member may be configured to wrap completely around the user's leg. An engagement element (e.g., hook and loop fasteners, etc.) may enable securing of the securing member to the leg. The securing member may comprise an adjustable strap, which may enable the length of the securing element to be tailored. The securing member may also include perforations, which may make the securing member more breathable and, therefore, more comfortable for a user to wear.

The drop foot brace may include a tensioning component that may be configured to removably couple with a support member that may be associated with a user's forefoot. The support member may comprise footwear (e.g., a shoe, boot, etc.) or a foot-receiving member that is configured to be worn on a user's forefoot. The tensioning component may, in one implementation, include at least one elongated element (e.g., a flexible element, such as a cord, a cable, etc.) and at least two support engagers (e.g., hooks, etc.).

The support engagers may be configured to engage eyelets or other features in an item of footwear (e.g., a shoe, a boot, etc.) or corresponding features of another embodiment of a foot-receiving member. Each support engager may be configured to move along a length of the elongated element and, in some embodiments, may include guides that are configured to prevent damage to the elongated element when the elongated element is under tension.

The tensioning component of a drop foot brace may also include a tightening element, which may be disposed on the exterior surface of the securing member. The tightening element may be configured to enable a user to selectively and controllably adjust the tension in the elongated element and, thus, pull the support engagers toward the securing member, as well as pull a support member that has been engaged by the support engagers closer to the securing member, all to exert an upward force on the forefoot of the user. The tightening may comprise a rotatable element, which may be configured to wind the elongated element about a spool.

Guides on the securing member of the drop foot brace may ensure that the elongated element is properly positioned, and that it remains properly positioned as the tension in the elongated element is adjusted. In a specific embodiment, a center guide may be disposed somewhat centrally on the securing member, towards a lower edge of the securing member. A lateral guide may also be disposed on the securing member, at a location that is lateral to (i.e., on the out-side of the foot and ankle) and higher than that of the center guide. A medial guide may be disposed on the securing member at a location that is medial to (i.e., on the in-side of the foot and ankle) and higher than that of the center guide, and opposite from the location of the lateral guide. Lateral and medial support engagers may be disposed on the elongated element, with the lateral support engager being positioned between the center guide and the lateral guide and the medial support engager being positioned between the center guide and the medial guide.

In certain embodiments, the foot-receiving member is a band configured to be disposed (e.g., wrapped, etc.) at least partially around a foot of a user and to couple with the tensioning component; for example, by being engaged by the lateral and medial support engagers. The band may be worn over a footwear item, such as a sandal or flip-flop, or it may be worn over a bare foot of a user. The foot-receiving member may also include a strap oriented transverse to the band and configured and oriented to fit between a user's toes to help maintain the position of the band.

Also disclosed herein is a system. In various embodiments, a system may include a foot-receiving member (e.g., an item of footwear, a band, etc.) and a drop foot brace. In a specific embodiment, where the foot-receiving member comprises an item of footwear, the footwear may be configured to be secured to a wearer's foot with laces. Such an item of footwear may include an upper with first second sides and a tongue disposed partially beneath and between the first and second sides. In use, laces are often secured between the first and second sides of the upper in a crisscross pattern over the tongue to secure the item of footwear to the foot. The footwear item may be a specialized footwear item designed for use with the drop foot brace, or may be a standard item of footwear.

The drop foot brace of such a system may include a securing member that comprises a cuff that is configured to encircle a portion of a leg of a user between the user's ankle and his or her knee. A tensioning component carried by the cuff (e.g., an elongated element, support engagers, guides, etc.) is configured to removably couple to at least a first lace guide (e.g., a hole, an eyelet, a hook, a loop, etc.) on the first side of upper and a second lace guide on the second side of the upper. A tightening element, which may be carried by (e.g., attached to, etc.) the cuff and coupled to the tensioning component may include a spool and a control for winding the tensioning component about the spool to adjust (e.g., apply, release, etc.) tension on the tensioning component.

Further disclosed herein is a method for bracing a drop foot. The method may include attaching a securing member, such as a cuff, to a leg of an individual drop foot brace above the ankle and below the knee. The securing member may be oriented such that a tightening element is outwardly disposed on a forward facing surface of the securing member (e.g., over a user's shin, etc.). The method may also securing a foot-receiving member to a user's foot, as well as and coupling an elongated element of a tensioning component of the drop foot brace, which elongated element is associated with to the tightening element, to the foot-receiving member. A control of the tightening element may be actuated in a manner that increases tension within the tensioning component (e.g., causes the tensioning component to wind around a spool of the tightening element, etc.) until a desired amount of tension has been applied to the elongated element and a desired amount of force has been applied to the foot-receiving member (e.g., when the leg and the foot are oriented at a desired angle (e.g., about 90°, etc.) to one another.

Other aspects, as well as the features and advantages of various aspects, of the disclosed subject matter will become apparent to those of ordinary skill in the art from the ensuing description, the accompanying figures, and the appended claims.

### BRIEF DESCRIPTION OF THE DRAWINGS

In the Figures:

FIG. 1 is a front view of an exterior surface of an embodiment of an orthotic device for addressing foot drop, or “drop foot device”;

FIGS. 2A through 2C are views of an embodiment of an engagement element that may be used with device drop foot device;

FIG. 3 illustrates use of the embodiment of drop foot device shown in FIG. 1 in conjunction with a foot-receiving member that is configured to be worn without a shoe;

FIG. 4 shows use of the embodiment of drop foot brace depicted by FIG. 1 in conjunction with a shoe with laces; and

FIG. 5 is a flow chart illustrating an embodiment of a method for using an embodiment of a drop foot brace in conjunction with a foot receiving member or a shoe.

### DETAILED DESCRIPTION

FIG. 1 illustrates one embodiment of a drop foot brace 100. The drop foot brace 100 provides support for the forefoot of the user, and can be used to treat foot drop and other conditions where the forefoot needs additional support.

The drop foot brace 100 may include a securing member 102 for securing the drop foot brace 100 to the leg of a user wearing the drop foot brace 100. Because the securing member 102 is configured to be secured to the leg of a user, its construction and the materials from which it is made may be configured to enable the securing member 102 to engage a user's leg in a desired manner. Thus, the securing member 102 may include flexible materials (e.g., fabrics, polymeric films, cushioning materials, etc.), rigid or somewhat rigid materials (e.g., plastic or metallic structural elements, form-fitting pieces, etc.) or combinations of flexible and rigid or somewhat rigid materials.

The securing member 102 includes an interior surface that faces inwardly toward the leg of the user when the securing member 102 is being worn, and an exterior surface that faces outwardly from the leg. In some embodiments, the securing member 102 may be configured to wrap entirely around the leg. In other embodiments, the securing member 102 may be configured to physically contact only portions of the leg, such as the front of the leg and the back of the leg.

The embodiment of securing member 102 shown in FIG. 1 is a cuff. Other types and embodiments of securing members 102 are also within the scope of the disclosed subject matter. In the embodiment depicted by FIG. 1, the securing member 102 comprises a band-like structure for surrounding a portion of the leg of the user of the drop foot brace 100. The securing member 102 may be made from a flexible material such as neoprene. The securing member 102 may also include stiff or rigid components. The securing member 102 may also include foam material or other padding to make the securing member 102 more comfortable when worn by the user. The securing member 102 may be configured to be worn against the skin of the leg and/or over an item clothing (e.g., hosiery, a sock, a pants leg, etc.).

The securing member 102 may include a center portion 106 that is configured to be positioned on the front of the leg when being worn (as shown in connection with FIG. 3 and FIG. 4), a lateral side 104, and a medial side 108 opposite from the lateral side 104. The securing member 102 may include fastener (e.g., a hook and loop fastener (such as VELCRO®), etc.) to secure the securing member 102 to itself and to the leg of the user. In one embodiment, the exterior surface of the medial side 108 carries a hook material 110, as shown in FIG. 1. An interior surface of the lateral side 104 may carry loop material (not seen in FIG. 1). The user, when securing the securing member 102, may wrap the medial side 108 around the leg, and then wrap the lateral side 104 around the leg such that the loop material at least partially overlaps the hook material 110.

While FIG. 1 illustrates a securing member 102 using hook and loop fasteners, other types of fasteners may be used instead of or in addition to hook and loop fasteners to secure the securing member 102 to a user's leg. The embodiment of securing member 102 shown in FIG. 1 also includes an optional strap 112 that at least partially secures the securing member 102 to the leg of the user. The strap 112 may be adjustable. It may include a securing mechanism comprising a male clip component 116 and a female clip component 114. The strap 112 may employ other forms of securing mechanisms, such as snaps, hook and loop fasteners, and others.

In one embodiment, the strap 112 (or at least a central portion thereof) may comprise a chlorosulfonated polyethylene (CSPE) synthetic rubber material (CSM), such as HYPALON®. However, other appropriate materials may also be used. Such a strap 112 may also include lateral portions that comprise nylon webbing that is sewn to the central portion of the strap 112.

In use, a user may position the securing member 102 around his or her leg, then secure the securing member 102 in place with the hook material 110 and the loop material. The strap 112 may then be adjusted to further tighten or secure the securing member 102 to the leg. The strap 112 may be pulled around the securing member 102 such that the strap 112 is disposed over (and around) a majority or all of the securing member 102, and encircles the user's leg. The user may then adjust the tightness of the strap 112 to further secure the securing member 102 in position.

While FIG. 1 shows an embodiment of a securing member 102 with a lateral side 104 and a medial side 108 that are not connected to each other, in other embodiments, the lateral side 104 and the medial side 108 join one another at a back of the securing member 102, such that the securing member 102 forms a unitary band. In embodiments where the securing member 102 comprises a unitary band, elasticity in at least a portion of the unitary band may enable the securing member 102 to engage and remain in place on the leg of a user. For example, the securing member 102 may comprise a unitary band formed from a flexible neoprene material that the user slides over his or her foot and onto his or her leg.

The securing member 102 may be configured to fit the lower leg of the user above the ankle and below the knee, a location that is often referred to as the “crus” or the “gaiter.” The center portion 106 of the securing member 102 may be situated proximally above the foot when being worn, as shown in FIG. 3 and FIG. 4. In certain embodiments, the center portion 106 is configured to fit an individual's leg directly above the ankle. In such an embodiment, a bottom edge of the center portion 106 may include a recess 118 for accommodating the top of an indivdual's foot when the securing member 102 is secured to the individual's lower leg. The recess 118, which comprise a concave curvature, may make the securing member 102 more comfortable for an individual to wear. In some embodiments, a recess 118 may enable use of the drop foot brace 100 with footwear (e.g., shoes, boots, etc.).

In some embodiments, a securing member 102 may include a number of perforations 140 that extend therethrough (e.g., completely through the securing member 102, from the interior surface to the exterior surface; partially through the securing member; etc.). The perforations 140 may impart the securing member 102 with breathability, which may make it more comfortable to wear (e.g., by minimizing perspiration that might otherwise be caused by the securing member 102, etc.). In addition or as an alternative to perforations 140, a securing member 102 may include apertures (e.g., openings in the lateral sides 104 and 108 of the securing member 102, etc.). A wicking material on the interior surface of the securing member 102 may draw moisture away from the skin of in individual wearing the drop foot brace 100.

The securing member 102 may include one or more cut lines 142. Each cut line 142 may be configured to enable an individual to adjust the length of the first lateral side 104 of the securing member 102 by providing a location at which the first lateral side 104 may be cut to remove unneeded, excess portions of the first lateral side 104 and, thus, to ensure that the securing member 102 is sized appropriately for the individual who will be wearing it. In various embodiments, each cut line 142 may be configured in a manner that will help prevent fraying or other undesirable wear of the first lateral side 104 of the securing member 102 as it is cut (e.g., each cut line 142 may comprise an elongated location that is molded or otherwise compressed, etc.) to an appropriate length.

The drop foot brace 100 may also include a tensioning component that removably couples to a foot-receiving member (e.g., footwear, such as a shoe, a boot, etc.; etc.) worn by the user that is using the drop foot brace 100. The tensioning component couples the securing member 102 of the drop foot brace 100 to the foot-receiving member. The term “couple,” as used herein, may refer to direct coupling with no intermediate components or indirect coupling. The tensioning component may directly couple to the foot-receiving member, or may indirectly couple to the foot-receiving member. In the embodiment shown in FIG. 1, the tensioning component is an elongated element 120 having two support engagers 122a and 122b attached thereto. Other types of tensioning component may be used in alternative embodiments.

The elongated element 120 and the two support engagers 122a and 122b may removably couple the securing member 102 to the foot-receiving member worn by the user. As used herein, the term “elongated element” refers to any of a variety of thin, flexible elements along which tensile forces may be applied and maintained to provide a desired amount of bracing force (e.g., a cord, a cable, a rope, a string, etc.). The elongated element 120 may comprise a single stranded element or it may include a plurality of filaments or strands that are associated with one another in a manner that imparts the elongated element 120 with strength and/or durability (e.g., it may comprise a plurality of twisted and/or woven strands, etc.). Any suitable material may be used to form the elongated element 120 (e.g., metal wire, polyethylene, an aramid, an acrylic, or any other suitable material). In embodiments where the elongated element 120 includes a plurality of filaments or strands, ends of the filaments or strands may be secured together in a manner that prevents fraying and, thus, dissociation of the filaments or strands from one another.

The drop foot brace 100 may also include a tightening element 150 disposed on the exterior surface of the securing member 102. In the illustrated embodiment, the tightening element 150 includes a spool and a control for winding the elongated element 120 about the spool and, thus, to adjust tension in the elongated element 120. Where the support engagers 122b and 122a are engage features of a foot-receiving member that are configured to receive the support engagers 122a and 122b (e.g., eyelets of footwear, etc.), the tightening element 150 may pull the top of the foot-receiving member and, thus, of a foot held by the foot-receiving member, towards the securing member 102. In this manner, the drop foot brace 100 can provide support for an individual's forefoot and prevent the foot from dropping when the wearer lifts his or her foot while walking. In a specific embodiment, the tightening element 150 may comprise a tensioning element of the type made by BOA Technology, Inc.

In use, the user may turn a control of the tightening element 150 that causes the tightening element 150 to wind the elongated element 120 about the spool. This action removes slack from the elongated element 120, and introduces tension into the elongated element 120. The tightening element 150 may include a uni-directional control that prevents unwinding of (and a reduction of tension in) the elongated element 120 unless the user actuates a separate control to enable unwinding of the elongated element 120. Thus, the user can control the tightening element 150 to tailor the amount of tension in the elongated element 120 and, thus, to provide his or her foot with a desired amount of support. When the user wants to remove the drop foot brace 100 or otherwise release tension in the elongated element 120, the user may use the tightening element 150 in a manner that causes the elongated element 120 to unwind or that otherwise releases the elongated element 120.

Use of a tightening element 150 that winds and unwinds to control the amount of tension in the elongated element 120 may be easy for an individual to use since it requires less dexterity than many other tensioning systems. Such a tightening element 150 may be particularly useful where it is used by an individual who has suffered a stroke or other condition that affects his or her dexterity.

The elongated element 120 may have first and second ends that removably connect to the spool of the tightening element 150, enabling the elongated element 120 to be removed from the drop foot brace 100 without removing the tightening element 150. Removability of the elongated element 120 may be desirable for circumstances when replacement of the elongated element 120 is desired, such as when the elongated element 120 is worn or damaged.

FIG. 1 further illustrates guides that are used to position and orient the elongated element 120 relative to the drop foot brace 100 and the foot-receiving member. As used herein, the term “guide” refers to a structure that helps guide and/or position the elongated element 120. FIG. 1 illustrates an embodiment of drop foot brace 100 that includes three guides, a lateral guide 160a, a medial guide 160b, and a center guide 160c disposed on the center section 106 of the securing member 102.

In the center guide 160c is disposed on the center portion 106 of the securing member 102, at or adjacent to its lower edge, which is positioned closest to a user's foot when the drop foot brace 100 is worn by the user. The elongated element 120 passes through the center guide 160c. In FIG. 1, one support engager 122b is on one side of the center guide 160c, while the other support engager 122a is on the other, opposite side of the center guide 160c. In the depicted embodiment, the center guide 160c comprises a loop of flexible material; however, other configurations of a center guide 160c may be used.

The drop foot brace 100 may also include a lateral guide 160a disposed on the center portion 106 of the securing member 102 adjacent to the lateral side 104 of the center portion 106. The lateral guide 160a may be situated above (at a higher elevation on the center portion 106 than) the center guide 160c and, in some embodiments, adjacent to an upper edge of the center portion 106 of the securing member 102. The lateral guide 160a may comprise a reinforced loop of flexible material, as shown in FIG. 1, or it may have any other appropriate configuration.

The drop foot brace 100 may include a similar medial guide 160b disposed on the center portion 106 adjacent to a medial side 108 of the center portion 106 and at an elevation above that of the center guide 160c. The medial guide 160b may be disposed on an opposite side of the center portion 160 from the lateral guide 160a Like the lateral guide 160a, the medial guide 160b may comprise a reinforced loop of flexible material, or it may have any other suitable configuration.

As seen in FIG. 1, the elongated element 120 may extend from the tightening element 150 through the lateral guide 160a, then through the support engager 122b, which may be situated on the elongated element 120 between the lateral guide 160a and the center guide 160c. The elongated element 120 may then extend through the center guide 160c, into the support engager 122a, through the medial guide 160b and back into the tightening element 150.

The lateral guide 160a, medial guide 160b, and center guide 160c control the position of the elongated element 120 as the user uses the tightening element 150 to adjust the tension in the elongated element 120. In the configuration shown in FIG. 1, the lateral guide 160a, medial guide 160b, and center guide 160c orient the elongated element 120 when it is under tension such that the elongated element 120, when secured to a foot-receiving member by the support engagers 122a and 122b, lifts the forefoot of the user to make it easier for a person with weakness in the foot who is wearing the drop foot brace 100 to walk. Other configurations and/or arrangements of guides for the elongated element 120 that differ from those shown in FIG. 1 may also be used.

Each of FIGS. 2A-2C shows a different view of an embodiment of a support engager 122 that may be used for the drop foot brace 100. Of course, other embodiments of a support engager 122 that differ from the embodiment shown in FIGS. 2A-2C may also be used. FIG. 2A shows a three-quarter view of the support engager 122. The depicted support engager 122 includes an enlarged head 208 with a pair of divergently oriented flanges protruding from an upper end thereof and an aperture 206 extending laterally therethrough, from one side to the other. A vertically oriented elongated recess 210 (which, in the depicted embodiment, has a shape that resembles a teardrop) is located at each end of the aperture and extends into the side or lower surface of each flange. A neck 202 extends from the enlarged head 208 to a curved element, which forms a hook 204.

The hook 204 of the support engager 122 is configured to be coupled to a corresponding feature of the foot-receiving member (e.g., an eyelet, hook, loop, lace, etc., of a shoe or other footwear, etc.). Thus, when the hook 204 engages a corresponding feature of the foot receiving member, it (along with the remainder of the support engager 122 and the elongated element 120 and other features of the tensioning component) provides the necessary connection between the securing member 102 and the foot-receiving member of the drop foot brace 100. The hook 204 may, as shown, terminate in a raised lip to impart it with a “J” shape. A raised lip may help reduce the likelihood that the support engager 122 slips and accidentally uncouples from the feature of the foot-receiving member that the hook 204 engages.

In some embodiments, the support engage 122 may lack a curved hook 204, as other configurations may suitably engage corresponding features of a foot-receiving member.

The support engager 122 may be coupled to a foot-receiving member at a location adjacent to the distal end, or front, of a user's foot. Alternatively, the support engager 122 may engage another portion of a foot-receiving member, such as the sidewall of an item of footwear. The particular coupling options may vary based on the type of foot-receiving member and user preference.

The aperture 206 through the enlarged head 208 of the support engager 122 may be configure to receive the elongated element 120 shown in FIG. 1 and described in reference thereto. The aperture 206 may be sized and shaped to allow the support engager 122 to move along the length of the elongated element 120. In other embodiments, the support engager 122 may be fixed to a certain position along the length of the elongated element 120.

The enlarged head 208 of the support engager 122 may also define a guide, which is also referred to herein as a “hook guide” to distinguish it from the lateral guide 106a, the medial guide 106b, and the center guide 106c on the securing member 102, as discussed in reference to FIG. 1. In the depicted embodiment, the divergent flanges that protrude from the enlarged head 208 of the support engager 122, the recesses 210 in the outer (or lower) surfaces of the flanges and the aperture 206 define the hook guide. The hook guide urges the elongated element 120 toward the securing member 102 of the drop foot brace 100 when the elongated element 120 is under sufficient tension. More specifically, the hook guide and, more specifically, the recesses 210 and the angles at which their respective flanges are oriented may control the angle at which the elongated element 120 is bent when the elongated element 120 is under sufficient tension. In some embodiments, the hook guide may provide a substantially semi-circular path for the elongated element 120. In certain embodiments, the elongated element 120 may comprise a material that may be irreversibly deformed when subjected to tensile stress that exceeds a critical threshold. The hook guide may be configured to distribute the force applied to the elongated element 120 and increase or maximize the distance over which the elongated element 120 contacts the support engager 122. In this manner, the hook guide may prevent the elongated element 120 from exceeding its elastic and, thus, prevent or reduce permanent deformation of or other damage to the elongated element 120. Such a configuration of hook guide may also prevent bending of the elongated element 120 at sharp angles, which may reduce the possibility that the elongated element 120 will break or snap at any location where it contacts the support engager 122.

FIG. 3 illustrates an embodiment of a drop foot brace 100 being worn by a user and connected to a foot-receiving member. The foot-receiving member shown in FIG. 3 comprises a band 302 that wraps around at least a portion of the foot of the user. The band 302 includes a bottom surface (not seen) for receiving the plantar portion of the foot of the user, and an upper surface 320 that is configured to be positioned over the dorsal, or top, portion of the foot of the user, as seen in FIG. 3. The band 302 also includes a proximal opening 324 for receiving the user's foot, and a distal opening 322 through which the distal end 312 (e.g., toes, etc.) of the user's foot may protrude. In other embodiments, the band 302 may lack a distal opening 322 and, thus, it may be configured to cover the toes of the user.

The bottom surface of the band 302 may be made from a different material than the upper surface 320 of the band 302. For example, the bottom surface may include a durable tread to provide traction when the user is walking without any footwear or other protection on the band 302, or that helps prevent an item of footwear (i.e., a sandal, etc.) worn over (or under) the band 302 from slipping off of the foot.

The band 302 may be adjustable to accommodate a variety of different sizes of feet. As an example, the upper surface 320 of the band 302 may be configured to adjust to the size of a foot. Without limitation, the upper surface 320 of such a band 302 may a length adjustable element (e.g., a strap, etc.). The band 302 may be configured to fit over the bare foot of the user, or over a footwear item of the user; for example, the band 302 may fit over a flip-flop, a sandal, or other item of footwear worn on the foot of the user. Such an embodiment may be desirable where the footwear does not include lace guides that can be engaged by the support engagers 122a and 122b. In certain embodiments, the band 302 may be placed on the foot first, then the footwear item may be positioned over the band 302, as shown in FIG. 3.

Some embodiments of band 302 may include a strap 304 that removably secures the bottom surface to the upper surface 320. The strap 304 may partially close the distal opening 322 to help secure the band 302 to the foot of the user. In the embodiment shown, the strap 302 is configured and positioned to pass between the hallux (or big toe) and the second toe of the user.

The band 302 may include one or more connection points or other features to which the support engagers 122a and 122b (or other variety of tensioning component) may connect. The connection points may be eyelets, lace guides, hooks, loops, or any other suitable connection point. The connection points may be positioned on opposite sides of the band 302.

FIG. 4 illustrates an embodiment of a drop foot brace 100 being worn by a user and connected to an item of footwear 402. The footwear 402 may be a shoe, a boot, or any other type of footwear. The footwear 402 may have a high-top configuration, a mid-top configuration, or other. While the depicted embodiment shows the drop foot brace 100 as being separate from the footwear 402, in some embodiments, the drop foot brace 100 may be integral with the footwear 402. For example, the tightening element 150 may be built into the tongue 410 or another portion of the footwear 402 (e.g., in embodiments where the footwear 402 has a sufficiently high top, etc.). In such an embodiment, the securing member 102 may be defined by sides 404 and 406 of an upper of the footwear 402 and the tongue of the footwear 402.

In addition to sides 404 and 406, the upper of the footwear 402 may include a toe portion 408, an ankle portion 412 and a lacing area 420 between the sides 404 and 406. Lace guides 422 are positioned on opposite sides of the lacing area 420. The lace guides 422 may be eyelets, hooks, loops, or any other variety of lace guide 422. The footwear 402 includes a lace or other securing element that passes over the lacing area 420 and through the lace guides 422. The lace may be tightened to secure the footwear 402 to the foot of a user.

The user may attach the securing member 102 to the leg just above the footwear 402. The support engagers 122a and 122b may be sized to removably couple to the lace guides 422 of the footwear 402. The first support engager 122a may couple to a lace guide 422 on one side of the lacing area 420 and the second support engager 122b may couple to a lace guide 422 on the opposite side of the lacing area 420, as shown in FIG. 4. The user may couple the support engagers 122a and 122b to any lace guide 422 between the ankle portion 412 and the toe portion 408 of the footwear. The user may choose the lace guides 422 to which the support engagers 122a and 122b connect based on one or more of the footwear 402, the desired level of comfort, the desired amount of support, or any other criteria. While FIG. 4 shows support engagers 122a and 122b connecting to lace guides 422, the support engagers 122a and 122b may connect to any suitable feature of the footwear 402.

In another embodiment, the drop foot brace 100 may not use support engagers 122 as part of the tensioning component; for example, in one embodiment, the drop foot brace 100 may include a strap that connects to the elongated element 120. The strap may fit under the sole of the footwear 402 at the toe portion 408 of the footwear 402. In another embodiment, the tensioning component includes a toe cap that fits over at least part of the toe portion 408 of the footwear 402. The toe cap may connect to the elongated element 120 and thus allow the user to tension the elongated element 120 using the tightening element 150 such that the foot is properly supported.

FIG. 5 shows an embodiment of a method 500 for using a drop foot brace 100 (see FIGS. 1, 3 and 4). The method 500 may commence at reference 502 with attaching a securing member of a drop foot brace 100 to a leg of an individual. The drop foot brace 100 may be attached to the leg above the ankle and below the knee such that the tightening element 150 of the drop foot brace 100 is outwardly disposed on the surface of the securing member 102.

The method 500 may also involve, at reference 504, inserting the foot of the individual into a foot-receiving member. The foot-receiving member may be an item of footwear 402 (FIG. 4), a band 302 (FIG. 3), or other suitable foot-receiving member. The method 500 may further involve, at reference 506, connecting a tensioning component of the drop foot brace 100 to the foot-receiving member. The method may additionally involve connecting the tensioning component (e.g., the elongated element 102) to a tightening element 150 of the drop foot brace 100, the lateral, medial, and center guides 160a-c, and the support engagers 122a and 122b.

The method 500, at reference 508, may include tightening the elongated element 120. As a non-limiting example, a tightening element 150 may be used in a manner that causes the tensioning component to wind around a spool of the tightening element 150, etc.). The user may actuate the control by turning the control in a clockwise or counterclockwise direction. By winding the elongated element 120 around the spool, the user removes the slack from the connection between the tightening element 150 and the foot-receiving member.

The method 500 may also involve, at reference 510, releasing the control when the elongated element 120 is tensioned and maintains a desired angle (e.g., an approximately ninety-degree angle, etc.) between the foot and the leg. As a result, the foot of the user is provided with additional support. The user may then walk more comfortably despite issues with weakness in the foot.

The method 500 may involve other elements in addition to, or instead of, those shown in FIG. 5. For example, where the foot-receiving member is an item of footwear 402 (FIG. 4) with a plurality of lace guides and the tensioning component includes support engagers 122a and 122b, connecting the tensioning component of the drop foot brace 100 to the foot-receiving member may involve connecting the support engagers 122a and 122b to at least two lace guides on opposing lateral sides of the footwear 402. Where the foot-receiving member is a band 302 (FIG. 3) that is configured to wrap around at least a portion of the foot of the user and the tensioning component includes support engagers, connecting the tensioning component may involve connecting the support engagers to opposing lateral sides of the band 302. Where the securing member 102 includes a cuff, attaching the securing member 102 to a user's leg may involve securing the cuff around the leg of the individual at the crus. Other variations depending on the implementation are also within the scope of the present disclosure.

Although the foregoing description contains many specifics, these should not be construed as limiting the scope of any of the claims, but merely as providing illustrations of some embodiments of the disclosed subject matter. Similarly, other embodiments of the disclosed subject matter may be devised which do not depart from the spirit or scope of any of the claims. Features from different embodiments may be employed in combination. The scope of each claim is, therefore, indicated and limited only by its plain language and the legal equivalents thereto, rather than by the foregoing description. All additions, deletions and modifications to the disclosed subject matter that fall within the meaning and scope of any of the claims are to be embraced thereby. No element recited by any of the claims is intended to be a means-plus-function limitation unless the phrase “means for” is specifically used in that claim.

What is claimed:

1. A drop foot brace comprising:
a securing member comprising an interior surface and an exterior surface, the securing member configured to be secured to the drop foot brace to a leg of a user wearing the drop foot brace such that the interior surface faces inwardly toward the leg of the user;
a tensioning component that removably couples to a foot-receiving member for the user wearing the drop foot brace; and
a tightening element disposed on the exterior surface of the securing member, the tightening element comprising a spool and a control for winding the tensioning component about the spool to adjust tension on the tensioning component and pull the foot-receiving member toward the securing member.

2. The drop foot brace of claim 1, wherein the securing member comprises a cuff comprising a first lateral side and a second lateral side opposite the first lateral side.

3. The drop foot brace of claim 2, the first lateral side comprising loop material on the interior surface of at least a portion of the first lateral side, and the second lateral side comprising hook material on the exterior surface of at least a portion of the second lateral side, the loop material configured to engage the hook material such that the cuff can be secured to the leg of the user.

4. The drop foot brace of claim 3, wherein the securing member further comprises an adjustable strap comprising a male buckle component and a female buckle component, the strap encircling the cuff when the cuff is fitted to the leg of the user and further securing the cuff to the leg of the user.

5. The drop foot brace of claim 1, wherein the securing member comprises a center section that is situated above a proximal section of a foot of the user, the center section comprising a substantially-rounded indent for accommodating the proximal section of the foot of the user when the securing member is secured to the leg of the user at the ankle.

6. The drop foot brace of claim 1, the securing member further comprising a plurality of perforations through the interior surface to the exterior surface, the perforations providing breathability.

7. The drop foot brace of claim 1, the tensioning component further comprising an elongated element.

8. The drop foot brace of claim 7, the tensioning component further comprising a plurality of support engagers coupled to the elongated element, the support engagers configured to engage the foot-receiving member.

9. The drop foot brace of claim 1, further comprising the foot-receiving member, wherein the foot-receiving member comprises a band including: a bottom surface that situates on a plantar aspect of the foot of the user; an upper surface that situates on a dorsal aspect of the foot of the user; a proximal opening; and a distal opening.

10. The drop foot brace of claim 9, wherein the band fits over a footwear item of the user.

11. The drop foot brace of claim 9, wherein the band fits over a bare foot of the user.

12. The drop foot brace of claim 11, the foot-receiving member further comprising a strap coupled to the bottom surface and removably coupled to the upper surface, the strap partially closing the distal opening.

13. A system comprising:
an item of footwear comprising:
a lacing area between a first opposing side and a second opposing side of the footwear item, the lacing area extending from a toe portion of the footwear item to proximate an ankle portion of the footwear item;
a plurality of opposing lace guides positioned on opposing sides of the lacing area;
a drop foot brace comprising:
a cuff that encircles a portion of a leg of a user between an ankle and a knee;
a tensioning component that removably couples to at least a first lace guide on the first opposing side of the lacing area and a second lace guide on the second opposing side of the lacing area; and
a tightening element attached to the cuff and coupled to the tensioning component, the tightening element comprising a spool and a control for winding the tensioning component about the spool to place tension on the tensioning component.

14. The system of claim 13, wherein the footwear item is one of a shoe and a boot.

15. The system of claim 13, wherein the drop foot brace is integrally coupled with the footwear item.

16. The system of claim 13, wherein the tensioning component comprises a wire and at least a first support engager and a second support engager, the first support engager removably coupling to the first lace guide and the second support engager removably coupling to the second lace guide.

17. The system of claim 16, wherein the first support engager and the second support engager each comprises an aperture through which the wire passes and a recess for receiving the wire passing through the aperture and for orienting the wire towards the cuff of the drop foot brace when the wire is under tension.

18. A method for coupling a drop foot brace comprising:
attaching a securing member of a drop foot brace to a leg an individual wearing the drop foot brace above the ankle and below the knee such that a tightening element of the drop foot brace is outwardly disposed on the surface of the securing member;
inserting a foot of the individual into a foot-receiving member;
coupling a tensioning component of the drop foot brace, the tensioning component coupled to the tightening element, to the foot-receiving member;
actuating a control of the tightening element in a direction causing the tensioning component to wind around a spool of the tightening element; and
releasing the control when the tensioning component is tensioned and maintains an approximately ninety-degree angle between the leg and the foot.

19. The method of claim 18, wherein the foot-receiving member is a footwear item comprising a plurality of lace guides and the tensioning component comprises a plurality of support engagers, wherein coupling the tensioning component of the drop foot brace to the foot-receiving member comprises coupling the plurality of support engagers to at least two lace guides on opposing lateral sides of the footwear item.

20. The method of claim 19, wherein the foot-receiving member comprises a band that wraps around at least a portion of the foot of the user and the tensioning component comprises a plurality of support engagers, wherein coupling the tensioning component of the drop foot brace to the foot-receiving member comprises coupling the plurality of support engagers to opposing lateral sides of the band.
